(19) United States
(12) Patent Application Publication
(10) Pub. No.: US 2014/0219420 A1
(43) Pub. Date: Aug. 7, 2014

(54) X-RAY DIAGNOSTIC APPARATUS AND METHOD FOR CONTROLLING THE SAME

(71) Applicants: Kabushiki Kaisha Toshiba, Minato-ku (JP); Toshiba Medical Systems Corporation, Otawara-shi (JP)

(72) Inventors: Naobumi ISHIKAWA, Otawara-shi (JP); Koji AMIDA, Nasushiobara-shi (JP); Hayato KASAOKA, Nasushiobara-shi (JP)

(73) Assignees: Kabushiki Kaisha Toshiba, Minato-ku (JP); Toshiba Medical Systems Corporation, Otawara-shi (JP)

(21) Appl. No.: 14/249,879

(22) Filed: Apr. 10, 2014

Related U.S. Application Data
(63) Continuation of application No. PCT/JP2013/075510, filed on Sep. 20, 2013.

(30) Foreign Application Priority Data
Oct. 1, 2012 (JP) 2012-219343

(51) Int. Cl. A61B 6/00

(52) U.S. Cl. CPC A61B 6/54
USPC 378/62

(57) ABSTRACT

An X-ray diagnostic apparatus of an embodiment includes: an adjuster for adjusting an X-ray irradiation field of a radiographing unit; a range setter for setting an imaging range to be imaged by the radiographing unit; an interval setter for setting an imaging interval to be used when the radiographing unit images the imaging range; an area setter for setting a size of an image stitching area in which to stitch the X-ray images; an irradiation field acquirer for finding a target X-ray irradiation field of the radiographing unit by use of the imaging interval and the size of the image stitching area; and a controller for controlling the adjuster on the basis of the target X-ray irradiation field.

### CROSS-REFERENCE TO THE RELATED APPLICATION

This application is based on and claims the benefit of priority from International Application No. PCT/JP2013/075510, filed on Sep. 20, 2013 and Japanese Patent Application No. 2012-219343, filed on Oct. 1, 2012; the entire contents of which are incorporated herein by reference.

### FIELD

Embodiments described herein relate generally to an X-ray diagnostic apparatus and a method for controlling the same.

### BACKGROUND

An X-ray diagnostic apparatus is an apparatus configured to irradiate a subject on a bed with X-rays by using an X-ray irradiator, to detect an amount of X-rays having passed through the subject by using an X-ray detector, and thereby to produce and display images of an internal condition of the subject. The X-ray diagnostic apparatus is provided with an X-ray diaphragm unit, such as a collimator, for changing the X-ray irradiation field. The X-ray diaphragm unit, for example, includes: a pair of blades (slit plates) for blocking some of the X-rays; and a movement mechanism for moving the blades toward and away from each other. The X-ray diaphragm unit changes the X-ray irradiation field by adjusting the opening width (aperture of the diaphragm) between the pair of blades through which the x-rays pass.

One of radiographic measures using the above-described X-ray diagnostic apparatus is called long-length imaging, which is configured to produce a single X-ray image by stitching X-ray images together. The long-length imaging produces a single X-ray image representing a designated imaging range by: sequentially shooting two or more X-ray images in the designated imaging range at predetermined imaging intervals; and then stitching the X-ray images (shot images) together. Here, an operator starts shooting only after designating the imaging range, the imaging interval, the aperture of the diaphragm, a movement speed of the bed, and the like.

Nevertheless, when the imaging interval, the aperture of the diaphragm, the movement speed of the bend or the like is not set at a value optimal for the long-length imaging using the X-ray diagnostic apparatus, the shot images may lack continuity and part of the image in the imaging range generated by stitching the shot images together may be missing. In addition, condition settings vary depending on a region to be imaged, for instance, depending on imaging of a narrow imaging range at a short imaging interval or imaging of a wide imaging range at a long imaging interval. For this reason, every time the imaging takes place, it takes labor and time to set all the conditions such as the imaging interval, the aperture of the diaphragm, and the movement speed of the bed at the optimal values, and examination efficiency is therefore reduced.

### BRIEF DESCRIPTION OF THE DRAWINGS

FIG. 1 is a diagram showing a schematic configuration of an X-ray diagnostic apparatus of an embodiment.

FIG. 2 is a block diagram showing a schematic configuration of a control unit included in the X-ray diagnostic apparatus of the embodiment.

FIG. 3 is an explanatory diagram for explaining how the X-ray diagnostic apparatus of the embodiment calculates a target X-ray irradiation field.

FIG. 4 is a flowchart showing a flow of long-length imaging performed by the X-ray diagnostic apparatus of the embodiment.

### DETAILED DESCRIPTION

According to one embodiment, an X-ray diagnostic apparatus comprises: a radiographing unit configured to shoot an X-ray image of a subject while irradiating the subject with X-rays; an adjuster configured to adjust an X-ray irradiation field of the radiographing unit; an image generator configured to generate a single X-ray image by stitching a plurality of the X-ray images together; a range setter configured to set an imaging range to be imaged by the radiographing unit; an interval setter configured to set an imaging interval to be used when the radiographing unit images the imaging range; an area setter configured to set a size of an image stitching area in which to stitch the X-ray images; an irradiation field acquirer configured to find a target X-ray irradiation field of the radiographing unit by use of the imaging interval set by the interval setter and the size of the image stitching area set by the area setter; and a controller configured to control the adjuster on the basis of the target X-ray irradiation field found by the irradiation field acquirer.

According to another embodiment, provided is a method for controlling an X-ray diagnostic apparatus including a radiographing unit configured to shoot an X-ray image of a subject while irradiating the subject with X-rays, an adjuster configured to adjust an X-ray irradiation field of the radiographing unit, and an image generator configured to generate a single X-ray image by stitching a plurality of the X-ray images together. The method comprises the steps of: setting an imaging range to be imaged by the radiographing unit; setting an imaging interval to be used when the radiographing unit images the imaging range; setting a size of an image stitching area in which to stitch the X-ray images; finding a target X-ray irradiation field of the radiographing unit by use of the set imaging interval, and the set size of the image stitching area; and controlling the adjuster on the basis of the found target X-ray irradiation field.

Referring to the drawings, descriptions will be provided for an embodiment of the present invention.

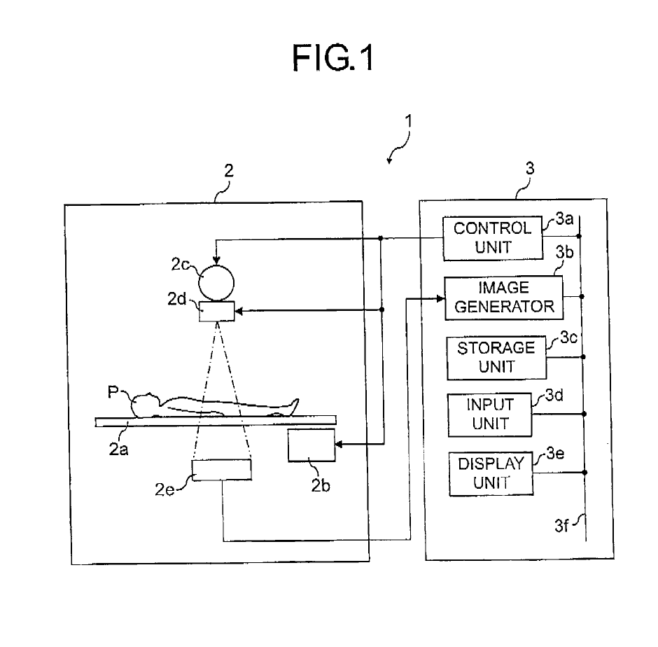

As shown in FIG. 1, an X-ray diagnostic apparatus 1 of the embodiment includes: a radiographing device 2 functioning as an image-taking device for taking X-ray images of a subject P; and a control device 3 for controlling the radiographing device 2. This X-ray diagnostic apparatus 1 is used to diagnose the spine, a leg and the like, for example.

The radiographing device 2 includes: a bed 2a for supporting the subject P such as a patient or a medical examinee; a movement driving unit 2b for moving the bed 2a; an X-ray irradiator 2c for irradiating the subject P on the bed 2a with X-rays; an X-ray diaphragm unit 2d for controlling the amount of X-rays emitted from the X-ray irradiator 2c; and an X-ray detector 2e for detecting the X-rays passing through the subject P on the bed 2a.

The bed 2a is a long-shaped tabletop on which the subject P lies down, and is formed in a way that makes the bed 2a movable in a longitudinal direction (in a body axis direction of the subject P on the bed 2a) and in a lateral direction (in a direction orthogonal to the body axis direction of the subject P on the bed 2a in a plane parallel to a supporting surface of the bed 2a) by the movement driving unit 2b. In addition, the bed 2a is formed in a way that makes the bed 2a turnable to a place where the subject P on the bed 2a is put in an upright position. The bed 2a is provided with shoulder rests for stabilizing the shoulders of the subject P, a footrest for supporting the subject P in the upright position, and the like depending on the necessity. The bed 2a is further provided with handgrips to be gripped by the subject P in the upright potion.

The movement driving unit 2b includes: a movement mechanism for moving the bed 2a in its longitudinal direction and the lateral direction; and a rise-fall mechanism (turning mechanism) for making the bed 2a rise and fall while supporting and turning the bed 2a. The movement driving unit 2b is electrically connected to the control device 3, and moves the bed 2a under the control of the control device 3.

The X-ray irradiator 2c is an X-ray tube for emitting X-rays onto the subject P on the bed 2a. The X-ray irradiator 2c is electrically connected to the control device 3, and irradiates the subject P on the bed 2a with the X-rays under the control of the control device 3. Here, the X-ray irradiator 2c, the X-ray diaphragm unit 2d, the X-ray detector 2e and the like are made rotatable together with the bed 2a while maintaining positional relations with one another.

The X-ray diaphragm unit 2d is an adjustor (a limiter for limiting the X-rays) for adjusting an X-ray irradiation field (irradiation range) by controlling the amount of the X-rays emitted from the X-ray irradiator 2c. The X-ray diaphragm unit 2d is electrically connected to the control device 3, and adjusts the irradiation field of X-rays on the subject P on the bed 2a, or the irradiation field of the X-rays on the X-ray detector 2e, under the control of the control device 3. Thereby, the X-rays emitted from the X-ray irradiator 2c are projected onto the given irradiation field on the subject P on the bed 2a with the amount of X-rays controlled by the X-ray diaphragm unit 2d.

In this respect, various types of X-ray diaphragm units may be used as the X-ray diaphragm unit 2d. For example, an X-ray diaphragm unit may be used in which: four X-ray blocking members made of lead or the like, for example, are assembled together in the shape of parallel crosses; the X-ray blocking members are moved toward and away from one another; and the position and size of the opening surrounded by the X-ray blocking members are changed as needed. In this case, the opening is a passage area of the X-rays while the rest of the X-ray diaphragm unit is a blocking area for absorbing and thus blocking the X-rays. Meanwhile, another X-ray diaphragm unit may be used in which: two X-ray blocking members are provided therein; the two X-ray are blocking members are moved toward or away from each other; and the position and size of a slit-shaped opening formed by the X-ray blocking members are changed as needed.

The X-ray detector 2e is opposed to the X-ray irradiator 2c and the X-ray diaphragm unit 2d with the bed 2a interposed in between, and is provided in a way that makes the X-ray detector 2e capable of detecting the X-rays emitted from the X-ray irradiator 2c and passing through the subject P on the bed 2a. The X-ray detector 2e is electrically connected to the control device 3, and sends the amount of detected X-rays (X-ray projection information) to the control device 3. An X-ray flat panel detector (FPD), for example, may be used as the X-ray detector 2e. Examples of a flat panel detector usable as the X-ray detector 2c includes; an indirect-conversion flat panel detector configured to indirectly convert the X-ray projection information into electric signals; and a direct-conversion flat panel detector configured to directly convert the X-ray projection information into electric signals.

The control device 3 includes: a control unit 3a for controlling the various components; an image generator 3b for generating various X-ray images by use of the X-ray projection information; a storage unit 3c for storing various programs and various data; an input unit 3d to be manipulated by an operator for the input operation; and a display unit 3e for displaying various images. The control unit 3a, the image generator 3b, the storage unit 3c, the input unit 3d and the display unit 3e are electrically connected to one another through a bus line 3f.

The control unit 3a controls the radiography by the radiographing device 2, in other words, the various components such as the movement driving unit 2b, the X-ray irradiator 2c and the X-ray diaphragm unit 2d, on the basis of the various programs and data which are stored in the storage unit 3c. In addition, the control unit 3a controls the displaying of the various images, such as the X-ray images, on the display unit 3e as well. A CPU (Central Processing Unit), for example, may be used as the control unit 3a.

The image generator 3b generates the X-ray images by performing various image processes, inclusive of a preparatory process and an image reconstruction process, on the X-ray projection information sent from the X-ray detector 2e. Furthermore, the image generator 3b generates a single X-ray image by performing an image process for stitching the X-ray images together. In this case, the X-ray images are a series of X-ray images sequentially shot in a designated imaging range at a predetermined imaging interval. The single X-ray image representing the designated imaging range is generated by stitching the shot images together.

The storage unit 3c is a storage device for storing the various programs and data, and stores the X-ray images, for example, as the various data. For instance, a hard disk (magnetic disk unit), a flash memory (semiconductor disk unit) and the like may be used as the storage unit 3c.

The input unit 3d is a manipulation unit for receiving input manipulations by the operator, which receives various input manipulations related to; various settings of a radiographic X-ray condition (X-ray irradiation condition), the imaging range, the imaging interval, and the like; and various instructions on the start and end of the imaging, the displaying of the images, and the like. Input devices such as a keyboard, a mouse, buttons, and levers may be used as the input unit 3d.

The display unit 3e is a display device for displaying various images inclusive of the X-ray images of the subject P, an image representing an operation panel, and the like. For example, a liquid crystal display, a CRT (Cathode Ray Tube) display, an organic EL (Electroluminescence) display and the like may be used as the display unit 3e.

Next, detailed descriptions will be provided for the control unit 3a.

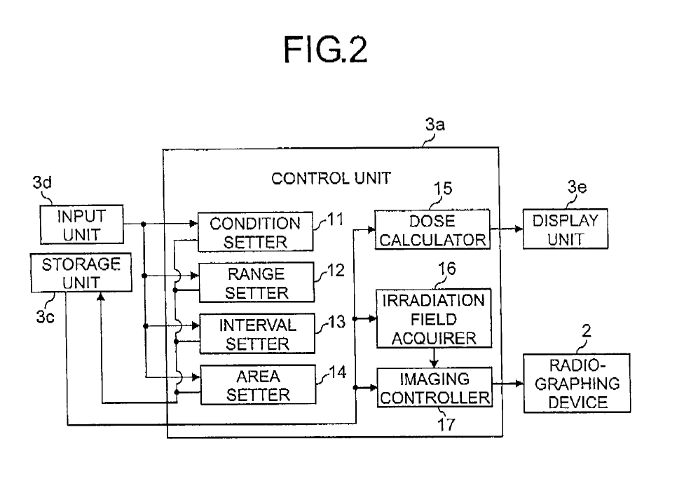

As shown in FIG. 2, the control unit 3a includes: a condition setter 11 for setting the radiographic X-ray condition (X-ray irradiation condition); a range setter 12 for setting the imaging range on the subject P; an interval setter 13 for setting the imaging interval for shooting the images in the imaging range; an area setter 14 for setting the size (dimensions) of an image stitching area defined by stitching the X-ray images together; a dose calculator 15 for calculating an estimated exposure dose to the subject P; an irradiation field acquirer 16 for finding a target X-ray irradiation field; and an imaging controller 17 for controlling the imaging by the radiographing device 2.

The condition setter 11 sets the radiographic X-ray condition (for example, the tube current, the tube voltage or the like) to be applied to the X-ray irradiator 2c in response to the operator's input manipulation on the input unit 3d, and stores the radiographic X-ray condition in the storage unit 3c. For example, the operator inputs the radiographic X-ray condition, such as the tube current, by manipulating the keyboard and the mouse of the input unit 3d.

The range setter 12 sets the imaging range on the subject P in response to the operator's input manipulation on the input unit 3d, and stores the imaging range in the storage unit 3c. For example, the operator specifies a position to start the imaging and a position to end the imaging by manipulating the buttons on the input unit 3d, and thereby inputs the imaging range between the two positions.

The interval setter 13 sets the imaging interval (of 3 cm, 5 cm, 10 cm, 20 cm, 30 cm or the like, for example) applied when shooting the images in the imaging range in response to the operator's input manipulation on the input unit 3d, and stores the imaging interval in the storage unit 3c. For example, manipulating the keyboard and the mouse of the input unit 3d, the operator directly inputs a value representing the imaging interval, or inputs the imaging interval by selecting a desired value from candidate values.

The area setter 14 sets the size of the image stitching area (image pasting area) with which to join (paste) the X-ray images in response to the operator's input manipulation on the input unit 3d, and stores the size thereof in the storage unit 3c. For example, manipulating the keyboard and the mouse of the input unit 3d, the operator such as a user or a service technician directly inputs the size of the image stitching area, or inputs the size of the image stitching area by selecting a desired value from candidate values. It should be noted that the image stitching area is an area in which the neighboring X-ray images overlap each other when stitched together.

The dose calculator 15 reads the radiographic X-ray condition, the imaging range and the imaging interval from the storage unit 3c, calculates the estimated X-ray exposure dose to the subject P by use of the read information, and sends the calculated estimated exposure dose to the display unit 3e. The display unit 3e receives the estimated exposure dose to the subject P which is sent from the dose calculator 15, and displays the estimated exposure dose.

The irradiation field acquirer 16 reads the imaging interval and the size of the image stitching area from the storage unit 3c, finds the target X-ray irradiation field by use of the read information, and sends the found target X-ray irradiation field to the imaging controller 17. Although a method of finding the target X-ray irradiation field in this procedure will be described later, the target X-ray irradiation field is automatically calculated by use of the set imaging interval and the set size of the image stitching area.

The imaging controller 17 receives the information on the target X-ray irradiation field sent from the irradiation field acquirer 16, and adjusts the X-ray irradiation field by controlling the X-ray diaphragm unit 2d by use of the received information. Thereafter, the imaging controller 17 reads the information on the imaging range, the imaging interval, and the like from the storage unit 3c, and controls the imaging by the radiographing device 2 by use of the read information. Incidentally, in order to adjust the X-ray irradiation field, the imaging controller 17 controls the aperture of the diaphragm of the X-ray diaphragm unit 2d in a way that makes the X-ray irradiation field applicable to the X-ray detector 2e coincides with the target X-ray irradiation field.

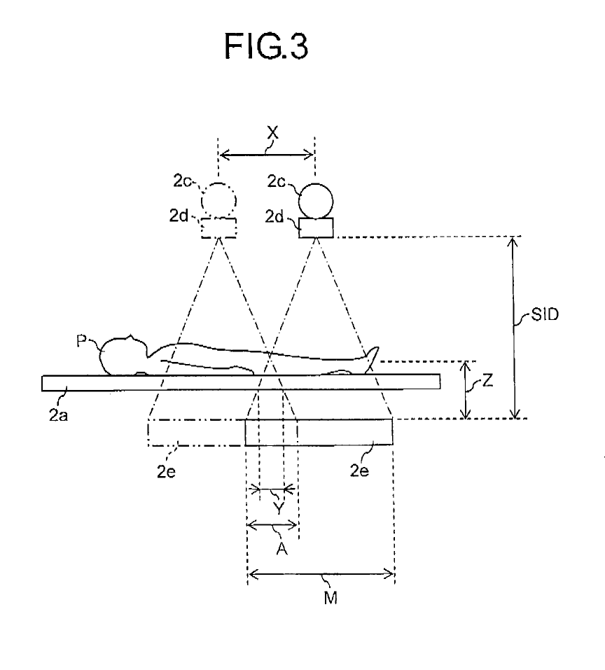

Here, referring to FIG. 3, descriptions will be provided for the method of finding the target X-ray irradiation field by using the irradiation field acquirer 16.

First of all, in FIG. 3, SID (Source-to-Image Distance) is a distance between a focal point of the X-ray irradiator 2c (an opening-side surface of the X-ray diaphragm unit 2d) and a detection surface of the X-ray detector 2e (a surface of the X-ray detector unit 2e facing the bed 2a). In addition, X denotes the imaging interval, and Y denotes an overlapping margin width (the width of the image stitching area in the body axis direction of the subject P). Z denotes a distance between the detection surface of the X-ray detector 2e and the body axis of the subject P. Although the distance Z is a variable value, the distance Z is preset by the input manipulation on the input unit 3d by the operator such as the user or the service technician. A denotes a maximum value of the overlapping margin width (A=M−X). Here, M denotes the maximum size of the field of view of the X-ray detector 2e.

In this case, the target X-ray irradiation field is defined as (X+Y)×SID/(SID−Z), where the overlapping margin width Y satisfies 0<Y≦(M−X)−(M×Z)/SID. The irradiation field acquirer 16 calculates the target X-ray irradiation field by substituting preset values of the imaging interval X, the overlapping margin width Y, the distance SID and the distance Z into the terms of this relational expression: the target X-ray irradiation field=(X+Y)×SID/(SID−Z), that is, a relational expression for calculating the irradiation field. In this respect, the target X-ray irradiation field is an irradiation field needed on the detection surface of the X-ray detector 2e.

It should be noted that the condition setter 11, the range setter 12, the interval setter 13, the area setter 14, the dose calculator 15, the irradiation field acquirer 16 and the imaging controller 17 may be made from hardware such as electrical circuits. Alternatively, they may be made from software such as programs for executing their respective functions. Otherwise, they may be made by combining hardware and software.

Next, descriptions will be provided for the long-length imaging process performed by the X-ray diagnostic apparatus 1.

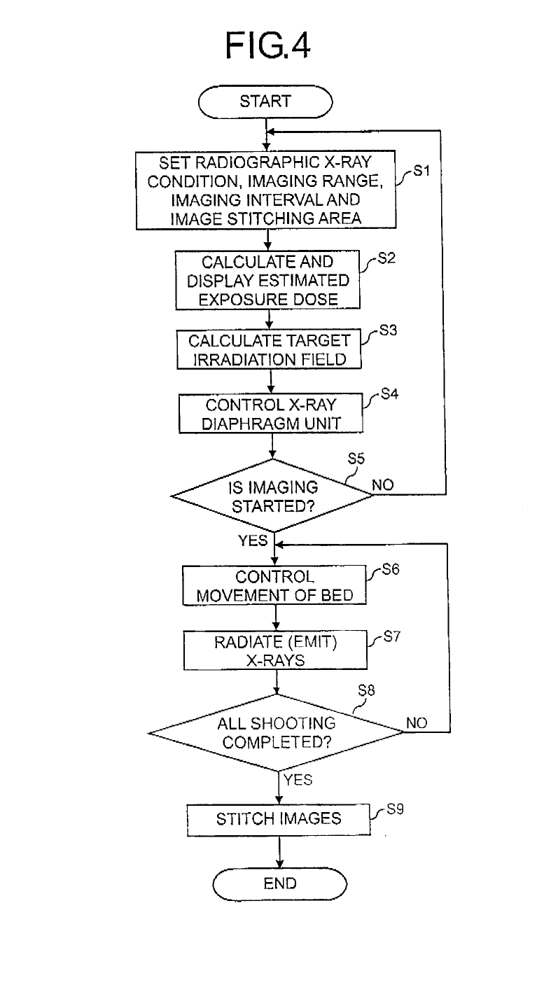

As shown in FIG. 4, first of all, the radiographic X-ray condition, the imaging range, the imaging interval and the image stitching area are set (step S1). They are set in response to the operator's manipulation on the input unit 3d.

To begin with, in setting the radiographic X-ray condition (X-ray irradiation condition), for example, the operator who is the user specifies a desired radiographic X-ray condition (for example, the tube current, the tube voltage or the like) by manipulating the input unit 3d. In accordance with this specification by means of the input manipulation, the condition setter 11 sets the specified radiographic X-ray condition in the storage unit 3c as a predetermined radiographic X-ray condition.

Furthermore, in setting the imaging range, for example, the operator who is the user moves the bed 2a while performing fluoroscopy, and specifies the position to start the imaging by pressing a button on the input unit 3d when the bed 2a reaches the position to start the imaging (as a manipulation for specifying the position to start the radiography). Thereafter, when the bed 2a reaches the position to end the imaging, the operator specifies the position to end the imaging by pressing the button on the input unit 3d again (as a manipulation for specifying the position to end the radiography). In response to the specifications given by these input manipulations, the range setter 12 sets a range from the position to start the imaging to the position to end the imaging in the storage unit 3c as a predetermined imaging range.

Moreover, in setting the imaging interval, for example, the operator who is the user specifies a desired imaging interval by selecting it from multiple candidates for the imaging interval which have been prepared beforehand (for example, 3 cm, 5 cm, 10 cm, 20 cm, 30 cm and the like) through the operator's input manipulation on the input unit 3d. In response to the specification given through this input manipulation, the interval setter 13 sets the specified imaging interval in the storage unit 3c as a predetermined imaging interval.

Another way to set the imaging interval is that the operator who is the user specifies a desired imaging interval by selecting a desired one from two imaging modes prepared beforehand, which include an imaging mode (first imaging mode) at a wide imaging interval (first imaging interval) and an imaging mode (second imaging mode) at a narrow imaging interval (second imaging interval) which is narrower than the first imaging interval, through the operator's input manipulation on the input unit 3d. In response to the instruction given through this input manipulation, the interval setter 13 sets the specified imaging interval in the storage unit 3c as a predetermined imaging interval. The imaging interval used in each imaging mode is represented by a variable value, which is changed, for example, by the input manipulation on the input unit 3d by the operator such as the service technician or the user.

What is more, in setting the image stitching area, for example, the operator such as the user or the service technician specifies a desired size of the image stitching area through the input manipulation on the input unit 3d. In response to the specification given through the input manipulation, the area setter 14 sets the specified size of the image stitching area in the storage unit 3c as a predetermined size of the image stitching area. This setting may be performed by the user during the medical examination, or by the service technician before the medical examination. The size of the image stitching area is a value which does not have to be re-set each time the imaging is performed if once set at an optimal value. Nevertheless, the size of the image stitching area can be re-set depending on the necessity.

In this respect, the foregoing settings may be automatically carried out in accordance with contents of a medical examination (for example, a bodily part to be examined). In this case, for example, candidates for the bodily part to be examined are listed and displayed, and the operator specifies a desired bodily part to be examined by selecting it from the candidates through the input manipulation on the input unit 3d. In response to the specification given through the input manipulation, the setters 11 to 14 set respective values corresponding to the specified bodily part in the storage unit 3c. Incidentally, the radiographic X-ray condition, the imaging range, the imaging interval, the size of the image stitching area, and the like are stored therein as predetermined values associated with each bodily part to be examined; and once the bodily part to be examined is specified in the above-described manner, the predetermined values associated with the bodily part to be examined are read and set in the storage unit 3c.

After the process in step S1, the dose calculator 15 calculates the estimated X-ray exposure dose by use of the radiographic X-ray condition, imaging range and imaging interval, and the calculated estimated exposure dose is displayed on the display unit 3e (step S2). In this case, the dose calculator 15 reads the radiographic X-ray condition, the imaging range and the imaging interval, which have been set in the storage unit 3c, from the storage unit 3c. Then, the dose calculator 15 calculates the estimated X-ray exposure dose from the read radiographic X-ray condition, the imaging range and the imaging interval, and displays the calculated estimated X-ray exposure dose. In this manner, the exposure dose before execution of the imaging (the estimated exposure dose) is automatically calculated on the basis of the radiographic X-ray condition, the imaging range and the imaging interval specified by the operator, and is thus presented to the operator.

After the process in step S2, the irradiation field acquirer 16 calculates the target X-ray irradiation field by use of the imaging interval and the size of the image stitching area (step S3). In this case, the irradiation field acquirer 16 calculates the target x-ray irradiation field, for example, by: reading the values of the imaging interval X, the overlapping margin width Y, the distance SID and the distance Z from the storage unit 3c; and substituting these values into the terms of the relational expression: the target X-ray irradiation field=(X+Y)×SID/(SID−Z).

After the process in step S3, the imaging controller 17 controls the X-ray diaphragm unit 2d such that the X-ray irradiation field applicable to the X-ray detector 2e coincides with the target X-ray irradiation field (step S4). Here, the imaging control unit 17 adjusts the aperture of the diaphragm of the X-ray diaphragm unit 2d so as to make the X-ray irradiation field applicable to the X-ray detector 2e coincide with the target X-ray irradiation field. Thus, the X-ray irradiation field applicable to the X-ray detector 2e automatically coincides with the target X-ray irradiation field, and the imaging can be started accordingly.

After the process in step S4, it is judged whether an imaging start instruction or a re-set instruction has been issued (step S5). If the re-set instruction has been issued instead of the imaging start instruction (if NO in step S5), the process returns to step S1. In step S5, the operator checks the imaging range, the imaging interval, the aperture of the diaphragm and the like, and instructs to start the imaging by pressing an imaging start button on the input unit 3d (as a manipulation for the imaging start instruction) if their values are satisfactory. On the other hand, if the values are not satisfactory, or if the imaging range, the imaging interval and the like need to be set again, the operator instructs re-setting by pressing a re-set button on the input unit 3d (as a manipulation for the re-set instruction).

If it is judged in step S5 that the imaging start instruction has been issued (if YES in step S5), the imaging controller 17 controls the movement of the bed 2a (in step S6). Each time the bed 2a moves to a position for shooting on the basis of the set imaging range and the set imaging interval, X-rays are radiated (emitted) upon arrival of the bed 2a at the shooting position (step S7).

Here, a relation between the movement of the bed 2a and the timing of the emission is set, for example, in a way that in a case where the imaging interval is equal to or greater than a predetermined value (in a case of the wide imaging interval), the movement speed of the bed 2a is increased and the emission is carried out with the movement of the bed 2a stopped upon its arrival at the shooting position; and in a case where the imaging interval is less than the predetermined value (in a case of the narrow imaging interval), the movement speed of the bed 2a is decreased and the emission is carried out without stopping the movement of the bed 2a on and after its arrival at the shooting position. In this manner, the movement speed of the bed 2a is controlled in accordance with the imaging interval, and the X-ray radiation (emission) is carried out when the bed 2a moves the distance of the imaging interval.

After the process in step S7, it is judged whether or not all the shooting in the imaging range (the imaging throughout the imaging range) has been completed (step S8). If it is judged that all the shooting in the imaging range has not been completed yet (if NO in step S8), the process returns to step S6 and the processes in steps S6, S7 are repeated. Thereby, the X-ray images are sequentially shot in the imaging range at the predetermined interval, and the multiple X-ray images are accordingly acquired.

If it is judged in step S8 that all the shooting in the imaging range has been completed (if YES in step S8), the multiple X-ray images shot in the imaging range are stitched together in accordance with the above-described size of the image stitching area, and the single X-ray image is thus generated (step S9). Thereafter, the single X-ray image is displayed on the display unit 3e, or stored in the storage unit 3c.

This imaging process automatically adjusts the aperture of the diaphragm of the X-ray diaphragm unit 2d at the optimal value once the operator sets the imaging range, the imaging interval and the like. For this reason, the user himself/herself no longer needs to adjust all the parameters, inclusive of the imaging interval and the aperture of the diaphragm, at their respective optimal values each time the user carries out the imaging. As a consequence, the efficiency of the medical examination improves. For example, it is difficult for the operator to adjust the aperture of the diaphragm of the X-ray diaphragm unit 2d at an opening which enables the images to be appropriately stitched together and inhibits unwanted radiation exposure, and it takes labor and time to do so. However, the automatic adjustment of the aperture of the diaphragm of the X-ray diaphragm unit 2d at the optimal value eliminates such a problem. Furthermore, since the various condition values can be set at the optimal values depending on the region to be shot such as the spine or a leg, it is possible to easily perform the long-length imaging depending on the region to be shot. Moreover, since the estimated exposure dose is displayed before the imaging, the user can grasp the estimated exposure dose and use the information on the estimated exposure dose for reducing the radiation exposure.

As described above, the embodiment finds the target X-ray irradiation field for the radiographing device 2 by use of the set imaging interval and the set size of the image stitching area, adjusts the X-ray irradiation field of the radiographing device 2 by use of the found target X-ray irradiation field, and causes the radiographing device 2 to perform the imaging by use of the set imaging range and the set imaging interval. Thereby, the X-ray irradiation field of the radiographing device 2 is automatically adjusted at the optimal value. For this reason, it is possible to prevent part of the images representing the imaging range from being missing when stitching the images together, and to obtain a favorable image representing the desired imaging range in the long-length imaging. What is more, since the X-ray irradiation field of the radiographing device 2 is automatically adjusted at the optimal value, it is possible to involve the user less labor and time, and accordingly to enhance the efficiency of the medical examination.

In addition, since the estimated exposure dose to the subject P is calculated by use of the set X-ray irradiation condition, the set imaging range and the set imaging interval, and the calculated estimated exposure dose to the subject P is displayed, the operator who is the user can grasp the estimated exposure dose before the imaging. This makes it possible to change the various settings if the estimated exposure dose falls outside the tolerable range, and resultantly to achieve a reduction in the radiation exposure.

Furthermore, since the radiographing device 2 performs the imaging by controlling the relative movement speed of the X-ray irradiator 2c and the X-ray detector 2e relative to the bed 2a to be moved by the movement driving unit 2b, on the basis of the set imaging range and the set imaging interval, the relative movement speed of the bed 2a relative to the X-ray irradiator 2c and the X-ray detector 2e is automatically adjusted. This makes it possible to obtain the favorable image representing the desired imaging range more reliably, and to enhance the efficiency of the medical examination more securely.

Moreover, since the radiographing device 2 performs the imaging by changing the relative movement speed of the X-ray irradiator 2c and the X-ray detector 2e relative to the bed 2a to be moved by the movement driving unit 2b in accordance with the set imaging interval, the relative movement speed can be controlled in accordance with the imaging interval. For example, in a case where the imaging interval is the first imaging interval, the relative movement speed of the bed 2a relative to the X-ray irradiator 2c and the X-ray detector 2e is set at a first relative movement speed, and the X-ray irradiator 2c performs its irradiation at the first imaging interval by stopping the relative movement of the bed 2a relative to the X-ray irradiator 2c and the X-ray detector 2e. Meanwhile, in a case where the imaging interval is the second imaging interval narrower than the first imaging interval, the relative movement speed of the bed 2a relative to the X-ray irradiator 2c and the X-ray detector 2e is set at a second relative movement speed which is slower than the first relative movement speed, and the X-ray irradiator 2c performs its irradiation at the second imaging interval while moving the bed 2a relative to the X-ray irradiator 2c and the X-ray detector 2e. Thereby, the relative movement speed and operational patterns are set at the respective optimal values through the automatic control. For this reason, it is possible to obtain the favorable image representing the desired imaging range yet more reliably, and to enhance the efficiency of the medical examination yet more securely.

In the embodiment, the bed 2a is moved relative to the X-ray irradiator 2c and the X-ray detector 2e. However, the present invention is not limited only to this configuration. For example, the X-ray irradiator 2c and the X-ray detector 2e may be moved relative to the bed 2a. Alternatively, the bed 2a and the set of the X-ray irradiator 2c and the X-ray detector 2e may be moved relative to each other.

Furthermore, although the embodiment is designed to move the bed 2a in the body axis direction of the subject P on the bed 2a, the present invention is not limited only to this configuration. For example, the X-ray irradiator 2c and the X-ray detector 2e may be moved in the body axis direction of the subject P on the bed 2a while fixing the bed 2a. Here, in addition to the body axis direction of the subject P on the bed 2a, the X-ray irradiator 2c and the X-ray detector 2e may be moved in a direction orthogonal to the body axis direction in a plane parallel to a supporting surface of the bed 2a. In this case, instead of sequentially shooting the X-ray images in the body axis direction of the subject P on the bed 2a, the single X-ray image may also be generated by sequentially shooting the X-ray images in the above-mentioned orthogonal direction and then stitching the X-ray images together.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray diagnostic apparatus comprising:
a radiographing unit configured to shoot an X-ray image of a subject while irradiating the subject with X-rays;
an adjuster configured to adjust an X-ray irradiation field of the radiographing unit;
an image generator configured to generate a single X-ray image by stitching a plurality of the X-ray images together;
a range setter configured to set an imaging range to be imaged by the radiographing unit;
an interval setter configured to set an imaging interval to be used when the radiographing unit images the imaging range;
an area setter configured to set a size of an image stitching area in which to stitch the X-ray images;
an irradiation field acquirer configured to find a target X-ray irradiation field of the radiographing unit by use of the imaging interval set by the interval setter and the size of the image stitching area set by the area setter; and
a controller configured to control the adjuster on the basis of the target X-ray irradiation field found by the irradiation field acquirer.

2. The X-ray diagnostic apparatus of claim 1, further comprising:
a condition setter configured to set an X-ray irradiation condition for the radiographing unit;
a dose calculator configured to calculate an estimated exposure dose to the subject by use of the X-ray irradiation condition set by the condition setter, the imaging range set by the range setter, and the imaging interval set by the interval setter; and
a display unit configured to display the estimated exposure dose to the subject calculated by the dose calculator.

3. The X-ray diagnostic apparatus of claim 1, wherein
the radiographing unit comprises:
a bed configured to support the subject;
an X-ray irradiator configured to irradiate the subject on the bed with the X-rays;
an X-ray detector configured to detect X-rays passing through the subject on the bed;
a movement driving unit configured to move the bed and a set of the X-ray irradiator and the X-ray detector relative to each other in a body axis direction of the subject on the bed, and
the controller controls a relative movement speed of the bed and the set of the X-ray irradiator and the X-ray detector to be relatively moved by the movement driving unit, on the basis of the imaging range set by the range setter and the imaging interval set by the interval setter.

4. The X-ray diagnostic apparatus of claim 2, wherein
the radiographing unit comprises:
a bed configured to support the subject;
an X-ray irradiator configured to irradiate the subject on the bed with the X-rays;
an X-ray detector configured to detect X-rays passing through the subject on the bed;
a movement driving unit configured to move the bed and a set of the X-ray irradiator and the X-ray detector relative to each other in a body axis direction of the subject on the bed, and
the controller controls a relative movement speed of the bed and the set of the X-ray irradiator and the X-ray detector to be relatively moved by the movement driving unit, on the basis of the imaging range set by the range setter and the imaging interval set by the interval setter.

5. The X-ray diagnostic apparatus of claim 3, wherein the controller changes the relative movement speed of the bed and the set of the X-ray irradiator and the X-ray detector to relatively be moved by the movement driving unit, in accordance with the imaging interval set by the interval setter.

6. The X-ray diagnostic apparatus of claim 4, wherein the controller changes the relative movement speed of the bed and the set of the X-ray irradiator and the X-ray detector to relatively be moved by the movement driving unit, in accordance with the imaging interval set by the interval setter.

7. The X-ray diagnostic apparatus of claim 5, wherein
in a case where the imaging interval is a first imaging interval, the controller sets the relative movement speed of the bed and the set of the X-ray irradiator and the X-ray detector at a first relative movement speed, and makes the X-ray irradiator perform irradiation at the first imaging interval by stopping the relative movement of the bed and the set of the X-ray irradiator and the X-ray detector, and
in a case where the imaging interval is a second imaging interval narrower than the first imaging interval, the controller sets the relative movement speed of the bed and the set of the X-ray irradiator and the X-ray detector at a second relative movement speed slower than the first relative movement speed, and makes the X-ray irradiator perform the irradiation at the second imaging interval while moving the bed and the set of the X-ray irradiator and the X-ray detector relative to each other.

8. The X-ray diagnostic apparatus of claim 6, wherein
in a case where the imaging interval is a first imaging interval, the controller sets the relative movement speed of the bed and the set of the X-ray irradiator and the X-ray detector at a first relative movement speed, and makes the X-ray irradiator perform irradiation at the first imaging interval by stopping the relative movement of the bed and the set of the X-ray irradiator and the X-ray detector, and
in a case where the imaging interval is a second imaging interval narrower than the first imaging interval, the controller sets the relative movement speed of the bed and the set of the X-ray irradiator and the X-ray detector at a second relative movement speed slower than the first relative movement speed, and makes the X-ray irradiator perform the irradiation at the second imaging interval while moving the bed and the set of the X-ray irradiator and the X-ray detector relative to each other.

9. The X-ray diagnostic apparatus of claim 1, further comprising an input unit to be used by an operator to perform input manipulation, wherein
the range setter sets the imaging range in accordance with the operator's input manipulation on the input unit,
the interval setter sets the imaging interval in accordance with the operator's input manipulation on the input unit, and
the area setter sets the size of the image stitching area in accordance with the operator's input manipulation on the input unit.

10. The X-ray diagnostic apparatus of claim 2, further comprising an input unit to be used by an operator to perform input manipulation, wherein
the range setter sets the imaging range in accordance with the operator's input manipulation on the input unit,
the interval setter sets the imaging interval in accordance with the operator's input manipulation on the input unit, and
the area setter sets the size of the image stitching area in accordance with the operator's input manipulation on the input unit.

11. A method for controlling an X-ray diagnostic apparatus provided with
a radiographing unit configured to shoot an X-ray image of a subject while irradiating the subject with X-rays,
an adjuster configured to adjust an X-ray irradiation field of the radiographing unit, and
an image generator configured to generate a single X-ray image by stitching a plurality of the X-ray images together,
the method comprising the steps of:
setting an imaging range to be imaged by the radiographing unit;
setting an imaging interval to be used when the radiographing unit images the imaging range;
setting a size of an image stitching area in which to stitch the X-ray images;
finding a target X-ray irradiation field of the radiographing unit by use of the set imaging interval, and the set size of the image stitching area; and
controlling the adjuster on the basis of the found target X-ray irradiation field.

12. The method for controlling an X-ray diagnostic apparatus of claim 11, further comprising the steps of:
setting an X-ray irradiation condition for the radiographing unit;
calculating an estimated exposure dose to the subject by use of the set X-ray irradiation condition, the set imaging range, and the Set imaging interval; and
displaying the calculated estimated exposure dose to the subject.

13. The method for controlling an X-ray diagnostic apparatus of claim 11,
wherein the radiographing unit comprises;
a bed configured to support the subject;
an X-ray irradiator configured to irradiate the subject on the bed with the X-rays;
an X-ray detector configured to detect X-rays passing through the subject on the bed;
a movement driving unit configured to move the bed and a set of the x-ray irradiator and the X-ray detector relative to each other in a body axis direction of the subject on the bed, and
the method further comprises the step of controlling a relative movement speed of the bed and the set of the X-ray irradiator and the X-ray detector to be moved by the movement driving unit, on the basis of the set imaging range and the set imaging interval.

14. The method for controlling an X-ray diagnostic apparatus of claim 12,
wherein the radiographing unit comprises:
a bed configured to support the subject;
an X-ray irradiator configured to irradiate the subject on the bed with the X-rays;
an X-ray detector configured to detect X-rays passing through the subject on the bed;
a movement driving unit configured to move the bed and a set of the X-ray irradiator and the X-ray detector relative to each other in a body axis direction of the subject on the bed, and
the method further comprises the step of controlling a relative movement speed of the bed and the set of the X-ray irradiator and the X-ray detector to be moved by the movement driving unit, on the basis of the set imaging range and the set imaging interval.

15. The method for controlling an X-ray diagnostic apparatus of claim 13, wherein in the step of controlling a relative movement speed, the relative movement speed of the bed and the set of the X-ray irradiator and the X-ray detector to be relatively moved by the movement driving unit is changed in accordance with the set imaging interval.

16. The method for controlling an X-ray diagnostic apparatus of claim 14, wherein in the step of controlling a relative movement speed, the relative movement speed of the bed and the set of the X-ray irradiator and the X-ray detector to be relatively moved by the movement driving unit is changed in accordance with the set imaging interval.

17. The method for controlling an X-ray diagnostic apparatus of claim 15, wherein in the step of controlling a relative movement speed,
in a case where the imaging interval is a first imaging interval, the relative movement speed of the bed and the set of the X-ray irradiator and the X-ray detector is set at a first relative movement speed, and the X-ray irradiator performs irradiation at the first imaging interval by stopping the relative movement of the bed and the set of the X-ray irradiator and the X-ray detector, and
in a case where the imaging interval is a second imaging interval narrower than the first imaging interval, the relative movement speed of the bed and the set of the X-ray irradiator and the X-ray detector is set at a second relative movement speed slower than the first relative movement speed, and the X-ray irradiator performs the irradiation at the second imaging interval while moving the bed and the set of the X-ray irradiator and the X-ray detector relatively to each other.

18. The method for controlling an X-ray diagnostic apparatus of claim 16, wherein in the step of controlling a relative movement speed,
in a case where the imaging interval is a first imaging interval, the relative movement speed of the bed and the set of the X-ray irradiator and the X-ray detector is set at a first relative movement speed, and the X-ray irradiator performs irradiation at the first imaging interval by stopping the relative movement of the bed and the set of the x-ray irradiator and the X-ray detector, and
in a case where the imaging interval is a second imaging interval narrower than the first imaging interval, the relative movement speed of the bed and the set of the X-ray irradiator and the X-ray detector is set at a second relative movement speed slower than the first relative movement speed, and the X-ray irradiator performs the irradiation at the second imaging interval while moving the bed and the set of the X-ray irradiator and the X-ray detector relatively to each other.

19. The method for controlling an X-ray diagnostic apparatus of claim 11, wherein
the X-ray diagnostic apparatus further comprises an input unit to be used by an operator to perform input manipulation,
in the step of setting an imaging range, the imaging range is set in accordance with the operator's input manipulation on the input unit,
in the step of setting an imaging interval, the imaging interval is set in accordance with the operator's input manipulation on the input unit, and
in the step setting a size of an image stitching area, the size of the image stitching area is set in accordance with the operator's input manipulation on the input unit.

20. The method for controlling an X-ray diagnostic apparatus of claim 12, wherein
the X-ray diagnostic apparatus further comprises an input unit to be used by an operator to perform input manipulation,
in the step of setting an imaging range, the imaging range is set in accordance with the operator's input manipulation on the input unit,
in the step of setting an imaging interval, the imaging interval is set in accordance with the operator's input manipulation on the input unit, and
in the step setting a size of an image stitching area, the size of the image stitching area is set in accordance with the operator's input manipulation on the input unit.
